(19) United States
(12) Patent Application Publication
(10) Pub. No.: US 2015/0129741 A1
(43) Pub. Date: May 14, 2015

(54) ARM SUPPORT APPARATUS

(71) Applicants: DENSO CORPORATION, Kariya-city (JP); SHINSHU UNIVERSITY, Matsumoto City (JP); TOKYO WOMEN'S MEDICAL UNIVERSITY, Tokyo (JP)

(72) Inventors: Hideki OKUDA, Nagoya (JP); Minoru TAKAHASHI, Tokoname-shi (JP); Kazuhiro HONGO, Matsumoto-shi (JP); Tetsuya GOTO, Matsumoto-shi (JP); Yosuke HARA, Matsumoto-shi (JP); Jun OKAMOTO, Tokyo (JP)

(21) Appl. No.: 14/538,903

(22) Filed: Nov. 12, 2014

(30) Foreign Application Priority Data
Nov. 12, 2013 (JP) 2013-233951

(51) Int. Cl. A61B 19/00; F16M 13/04

(52) U.S. Cl. CPC A61B 19/28; F16M 13/04
USPC 248/550; 248/118

(57) ABSTRACT

In an arm support apparatus, a grippable member is connected to a longitudinal mount and is located such that a part of the grippable member is grippable by a hand of an arm when a part of the arm is mounted on the longitudinal mount. The hand is located to be closer to the first end of the longitudinal mount than to the second end of the longitudinal mount. A second limiter limits movement of the part of the arm mounted on the longitudinal mount in both a first direction and a second direction. The first direction is away from the second end of the longitudinal mount in the longitudinal direction. The second direction is parallel to the lateral width of the longitudinal mount and perpendicular to the longitudinal direction.

### CROSS REFERENCE TO RELATED APPLICATIONS

This application is based on and claims the benefit of priority from Japanese Patent Application 2013-233951 filed on Nov. 12, 2013, the disclosure of which is incorporated in its entirety herein by reference.

### TECHNICAL FIELD

The present disclosure relates to arm support apparatuses that support an arm of an operator.

### BACKGROUND

For precise and/or long manual operations, such as neurosurgical operations, there are known arm support apparatuses for supporting an arm of an operator such as a doctor that performs surgical operations. An example of these arm support apparatuses is disclosed in Japanese Patent Application Publication No. H10-272163, referred to as a first patent publication. An arm support apparatus disclosed in the first patent publication is equipped with a movable multijoint arm having a mount portion at an end thereof. The forearm of an operator's arm is fixedly mounted on the mount portion with a belt, which allows the mount portion to follow motion of the supported arm. The arm support apparatus locks the movable multijoint arm when a foot switch is operated by the operator, thus preventing motion of the mount portion.

The arm support apparatus disclosed in the first patent publication necessitates unfastening and fastening the belt each time the operator's arm is dismounted from the mount portion for mount of instrument, such as tweezers, on a table. This may result in the operator's usability of the arm support apparatus deteriorating.

In order to address such a problem, there is known an arm support apparatus, which is disclosed in Japanese Patent Application Publication No. 2009-291363, referred to as a second patent publication.

The arm support apparatus disclosed in the second patent publication urges the mount portion upward from the lower side of the mount portion to bring the mount portion into contact with the forearm of an operator's arm based on friction force between the forearm and the mount portion. This permits the operator to easily mount a forearm on the mount portion or dismount a forearm from the mount portion while the multijoint arm is locked in motion.

### SUMMARY

However, there is at least one intermediate member, such as a cover or an operator's clothing, interposed between the mounted forearm and the mount portion in actual manual operations. For example, in actual surgical operations, a plastic film covering the mount portion, a non-woven fabric drape, and/or an operator's operation gown made from a non-woven fabric are interposed between the mounted forearm and the mount portion.

This reduces a friction coefficient defining the level of friction force between the forearm and the mount portion via the at least one intermediate member. In addition, if physiological water or blood was interposed between the mounted forearm and the mount portion, the friction coefficient might be also reduced.

Thus, there may be a need of a large urging force to reliably cause the mount portion to follow movement of the forearm based on the friction force between the forearm and the mount portion via the at least one intermediate member.

One aspect of the present disclosure therefore seeks to provide arm support apparatuses; each of the arm support apparatuses is capable of addressing the circumstances set forth above.

Specifically, an alternative aspect of the present disclosure aims to provide such arm support apparatuses, each of which is capable of causing a mount portion to follow motion of an operator's arm mounted on the mount portion stably and/or lightly without using a fastening member, such as a belt, for fastening the operator's arm on the mount portion.

According to an exemplary aspect of the present disclosure, there is provided an arm support apparatus for supporting an arm of an operator. The arm support apparatus includes a longitudinal mount on which a part of the arm of the operator is mountable. The longitudinal mount has a first end, a second end opposite to the first end in a longitudinal direction thereof, and a predetermined lateral width. The arm support apparatus includes a support member that has at least one joint and supports the longitudinal mount to be movable based on bending of the at least one joint. The arm support apparatus includes a first limiter that limits motion of the at least one joint to limit movement of the longitudinal mount when an operation mode of the arm support apparatus is set to a first mode. The first limiter releases limit of motion of the at least one joint to release limit of movement of the longitudinal mount when the operation mode of the arm support apparatus is set to a second mode. The arm support apparatus includes a biasing mechanism that applies biasing force to the longitudinal mount to bias the longitudinal mount upwardly when the operation mode of the arm support apparatus is set to the second mode. The arm support apparatus includes a grippable member connected to the longitudinal mount and located such that a part of the grippable member is grippable by a hand of the arm when the part of the arm is mounted on the longitudinal mount. The hand is located to be closer to the first end of the longitudinal mount than to the second end of the longitudinal mount. The arm support apparatus includes a second limiter that limits movement of the part of the arm mounted on the longitudinal mount in both a first direction and a second direction. The first direction is away from the second end of the longitudinal mount in the longitudinal direction. The second direction is parallel to the lateral width of the longitudinal mount and perpendicular to the longitudinal direction.

In the exemplary aspect of the present disclosure, in the second mode, because the biasing force is applied to the longitudinal mount upwardly, the biasing force serves to substantially balance the sum of the weight of the part of the arm mounted on the longitudinal mount and the weight of the longitudinal mount. This feature makes the longitudinal mount easily follow movement of the operator's arm in an upward direction or a downward direction. This results in maintenance of the mounted state of the part of the arm on the longitudinal mount.

In the exemplary aspect of the present disclosure, the second limiter limits movement of the part of the arm mounted on the longitudinal mount in both a first direction and a second direction. The first direction is away from the second end of the longitudinal mount in the longitudinal direction. The second direction is parallel to the lateral width of the longitudinal mount and perpendicular to the longitudinal direction.

This configuration of the second limiter makes the longitudinal mount follow movement of the operator's arm, a part of which is mounted on the longitudinal mount in the second direction, or a direction, which is referred to as a frontward direction, opposite to the first direction.

In addition, a grippable member is connected to the longitudinal mount and located such that a part of the grippable member is grippable by a hand of the arm when the part of the arm is mounted on the longitudinal mount. The hand is located to be closer to the first end of the longitudinal mount than to the second end of the longitudinal mount.

With this configuration, while the grippable member is gripped by the hand of the operator's arm, the operator moves the arm in the frontward direction. This causes the grippable member to pull the longitudinal mount in the frontward direction, resulting in the longitudinal mount following movement of the operator's arm in the same direction.

That is, in the second mode, the arm support apparatus makes it possible for the operator to pull the grippable member by the hand of the arm in the direction opposite to the first direction to cause the longitudinal mount portion to follow movement of the hand in the frontward direction. In the free mode, the arm support apparatus makes it possible for the operator to merely move the arm in the upward direction, the downward direction, the first direction, or the second direction to cause the longitudinal mount to follow movement of the arm in the same direction.

Thus, the arm support apparatus achieves an advantage of making the longitudinal mount follow lightly and stably movement of the operator's arm without the arm being fixed to the longitudinal mount even if the friction coefficient between the arm and the longitudinal mount is set to a lower value.

Various aspects of the present disclosure can include and/or exclude different features, and/or advantages where applicable. In addition, various aspects of the present disclosure can combine one or more feature of other embodiments where applicable. The descriptions of features, and/or advantages of particular embodiments should not be construed as limiting other embodiments or the claims.

### BRIEF DESCRIPTION OF THE DRAWINGS

Other aspects of the present disclosure will become apparent from the following description of embodiments with reference to the accompanying drawings in which:

FIG. 1 is a schematic view of an arm support apparatus according to a first embodiment of the present disclosure;

FIG. 2A is a perspective view schematically illustrating the outward appearance of an arm holder of the arm support apparatus according to the first embodiment;

FIG. 2B is a lateral cross sectional view of a bracket perpendicular to a longitudinal direction of a base of the arm holder;

FIG. 3 is a view schematically illustrating an example of the structure of a connecting wire illustrated in FIG. 2A;

FIG. 4 is a schematic view of an arm support apparatus according to a second embodiment of the present disclosure;

FIG. 5 is a flowchart schematically illustrating a mode determination task according to the second embodiment;

FIG. 6 is a mode transition view schematically illustrating how an operation mode of the arm support apparatus changes according to the second embodiment;

FIG. 7 is a schematic view of an arm support apparatus according to a third embodiment of the present disclosure;

FIG. 8 is a flowchart schematically illustrating a mode determination task according to the third embodiment;

FIG. 9 is a mode transition view schematically illustrating how an operation mode of the arm support apparatus changes according to the third embodiment; and

FIG. 10 is a perspective view schematically illustrating the outward appearance of an arm holder according to a modification of each of the first to third embodiments.

### DETAILED DESCRIPTION OF EMBODIMENT

Embodiments of the present disclosure will be described hereinafter with reference to the accompanying drawings. In the drawings, identical reference characters are utilized to identify identical corresponding components. In each of the following embodiments, there is described an arm support apparatus for supporting an arm, especially a forearm, of a doctor as an example of operators who performs medical operations, such as surgical operations. However, arm support apparatuses according to the present disclosure can be designed to support an arm of an operator who performs precise and/or long operations during a process of, for example, manufacturing a machine, such as precision machines.

### First Embodiment

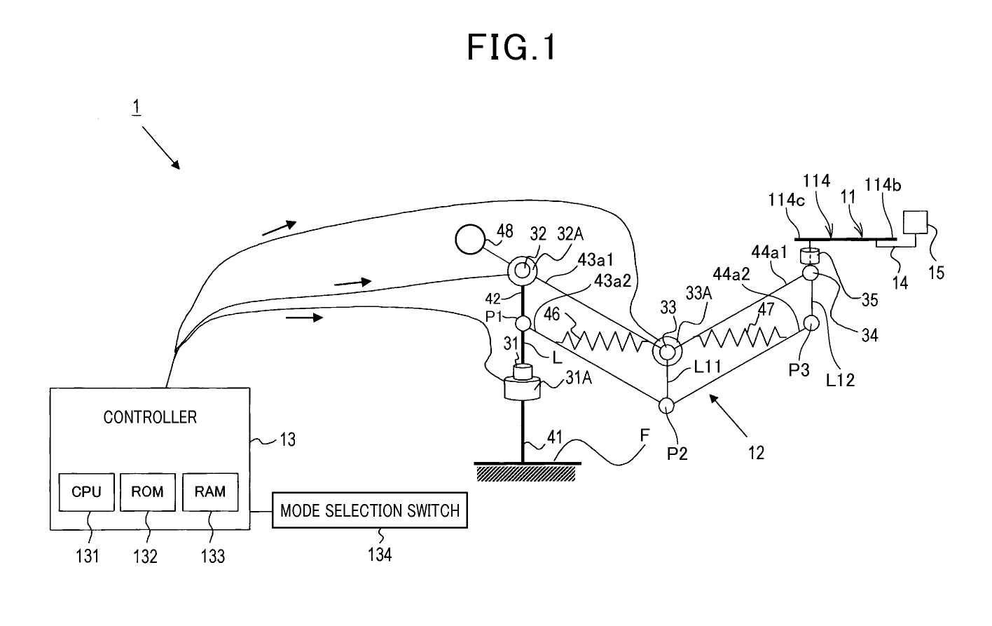

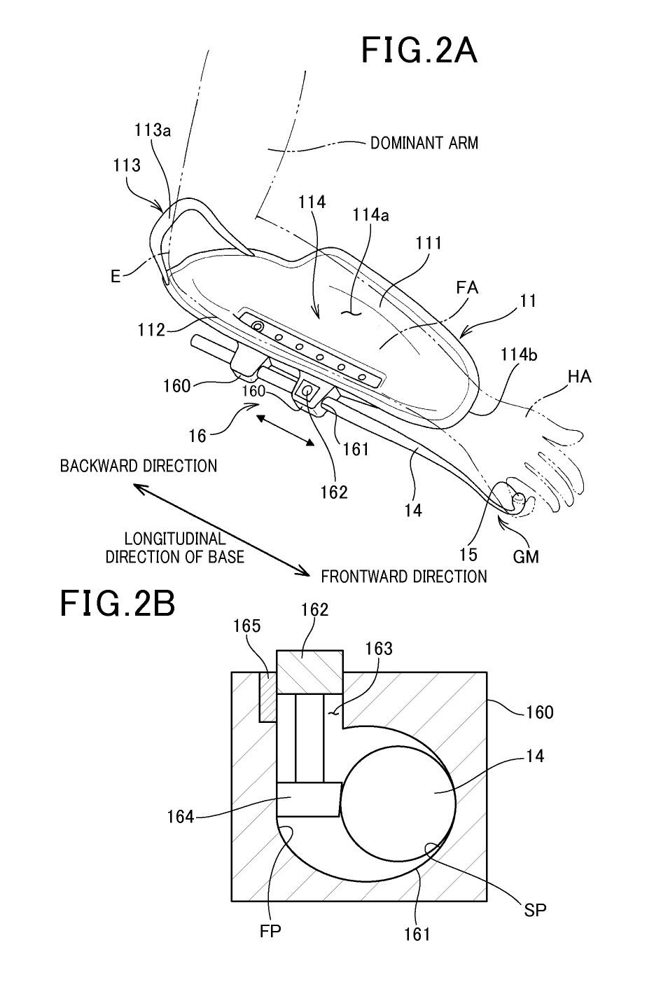

FIG. 1 illustrates a schematic view of an arm support apparatus 1 according to the first embodiment of the present disclosure, and FIG. 2A illustrates the outward appearance of the arm support apparatus 1.

Referring to FIGS. 1 and 2A, the arm support apparatus 1 is equipped with an arm holder 11, a multijoint arm 12, and a controller 13.

The arm holder 11 is operative to hold an arm A, particularly, the forearm FA, of the dominant arm of a doctor.

The multijoint arm 12, serving a support member, is designed to movably support the arm holder 11 according to external force applied to the arm holder 11. Specifically, the multijoint arm 12 has plural rotational joints that provide plural degrees of freedom.

The controller 13 is communicably connected to the multijoint arm 11 and is operative to control an operation mode of the arm support apparatus 1.

Referring to FIG. 2, the arm holder 11, which serves as a longitudinal mount, has a substantially longitudinal ellipsoidal base 114 with a top surface 114a on which the forearm FA of the dominant arm of a doctor is mountable; the top surface 114a will be referred to as a mount surface. The base 114 has a predetermined lateral width.

Note that the forearm FA of the dominant arm of a doctor can also be simply referred to as the forearm FA hereinafter.

The arm holder 11 also has a pair of sidewalls 111 and 112 extending upwardly from longitudinal sides of the base 114, so that the arm holder 11 has a substantially U-shape in its lateral cross section. The sidewalls 111 and 112 serve to support the forearm FA mounted on the mount surface 114a of the base 114 from respective sides of the forearm FA.

The base 114 has a first end 114b in its longitudinal direction and a second end 114c opposite to the first end 114b, so that the forearm FA is mountable while the elbow E is located on the second end 114c of the mount surface 114a of the base 114. Note that, in the first embodiment, a first side of the longitudinal direction of the base 114 outwardly away from the first end 114b will be referred to as a frontward direction, and a second side of the longitudinal direction of the base 114 outwardly away from the second end 114c will be referred to as a backward direction.

The arm holder 11 includes an elbow-position limiter 113 made from, for example, a high-rigidity member. The elbow-position limiter 113 includes an arched portion, i.e. a substantially U-shaped portion. Both ends of the U-shaped portion are attached to the base 114 across the second end 114c such that the U-shaped portion 113a is located above the second end 114c of the base 114 to form a support ring in which a part of the elbow E is fittable.

Specifically, when the forearm FA is mounted on the mount surface 114a, the hand HA of the dominant arm is located to be closer to the first end 114b of the base 114 than to the second end 114c of the base 114. In other words, the hand HA projects from the first end 114b of the base 114. In the state of the forearm FA being mounted on the base 114 of the arm holder 11, a lower portion of the elbow E is mounted on the second end 114c of the mount surface 114a with an upper portion of the elbow E is fitted in the elbow-position limiter 113. This limits the forearm FA mounted on the mount surface 114a of the base 114 from moving outwardly away from the second end 114c in the longitudinal direction of the base 114 relative to the arm holder 11.

Note that the sidewalls 111 and 112 will be referred to as left and right sidewalls 111 and 112 based on the state that the forearm FA is mounted on the mount surface 114a of the base 114.

Each of the left and right sidewalls 111 and 112 is made from, for example, a high-rigidity member. The left and right sidewalls 111 and 112 limit the forearm FA mounted on the mount surface 114a of the base 114 from moving outwardly away from the base 114 in the lateral direction of the base 114. The lateral direction is parallel to the lateral width of the base 114 and perpendicular to the longitudinal direction of the base 114.

The left and right sidewalls 111 and 112 and the elbow-position limiter 113 can be made from a same high-rigidity material, or the left and right sidewalls 111 and 112 can be made from a high-rigidity material different from a high rigid material from which the elbow-portion limiter 113 is made.

In addition, the arm support apparatus 1 includes a grippable member GM including a grip 15 located to be grippable by a part of the hand HA of the forearm FA mounted on the mount surface 114a of the base 114.

Specifically, the grippable member GM includes a connecting wire 14 having one end to which the grip 15 is attached. The grippable member GM also includes a support member 16 attached to, for example, an outer surface of the right sidewall 112. The support member 16 supports the connecting wire 14 such that the grip 15 is located where a part of the hand HA of the dominant arm of a doctor can grip the grip 15. For example, the grip 15 is made from a flexible material, such as rubber, and is designed to have a shape easily graspable by a finger of the hand HA of the dominant arm of a doctor. For example, the arm support apparatus 1 according to the first embodiment is designed for doctors whose dominant arms are right arms.

Thus, the grip 15 is located to be adjacent to the little finger of the right arm of a doctor whose forearm FA is mounted on the mount surface 114a of the base 114, and is designed to have a substantially cylindrical shape that can be easily gripped by the little finger of the right arm of a doctor.

The connecting wire 14 is configured to be deformable according to external force applied thereto or to the grip 15, thus supporting the grip 15 such that the position of the grip 15 is changeable relative to the arm holder 11.

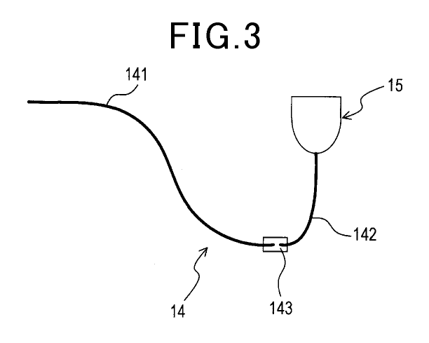

For example, referring to FIG. 3, the connecting wire 14 is comprised of a superelastic-alloy wire rod 141, a mild-steel wire rod, i.e. a wire, 142, and a joint member 143. The superelastic-alloy wire rod 141 has a first end and a second end opposite to the first end, and a predetermined length portion of the superelastic-alloy wire rod 141 including the first end is attached to the outer surface of the right sidewall 112 via the support member 16. The mild-steel wire rod 142 has a first end and a second end opposite to the first end. The second end of the superelastic-alloy wire rod 141 and the first end of the mild-steel wire rod 142 are joined to each other via the joint member 143 using, for example, caulking. The second end of the mild-steel wire rod 142 serves as the end to which the grip 15 is attached.

Note that superelastic-alloy members, such as super-alloy wire rods, are shape-memory alloys, such as alloys of titanium and nickel, each memorizing an original shape and having a transformation point equal to or lower than ordinary temperature. These superelastic members have a characteristic that, even if they are deformed when external force is applied thereto, they return to their original shapes when the external force is removed.

The superelastic-alloy wire rod 141 serves as an elastic deformation portion that is elastically deformed when an external force is applied to the grip 15. The mild-steel wire rod 142 serves as a deformation holding portion that, when an external force is applied to the mild-steel wire rod 142 so that it is deformed, holds the deformed shape. Specifically, as illustrated in FIG. 3, the length of the mild-steel wire rod 142 is shorter than the superelastic-alloy wire rod 141, so that a part of the connecting wire 14, which is adjacent to the grip 15, serves as the deformation holding portion, and the remaining part serves as the elastic deformation portion. This configuration permits an initial position of the grip 15 around the hand HA of the dominant arm of a doctor to be adjusted; this adjustment makes it possible for the little finger of the hand HA of the dominant arm of a doctor to easily grip the grip 15.

The connecting wire 14 is also configured not to expand in its length direction corresponding to the axial direction of the forearm FA mounted on the mount portion 114a of the base 114. This results in the connecting wire 14 being non-extendable in the front direction.

In other words, the length of the connecting wire 14 does not extend, which is different from the length of a material with a low level of hardness, such as rubber.

The support member 16 serves as an adjusting mechanism for adjusting the position of the grip 15 relative to the elbow-portion limiter 113, i.e. the elbow E of the forearm FA whose location is limited by the elbow-position limiter 113 in the longitudinal direction of the base 114.

FIG. 2B schematically illustrates an example of the structure of the adjusting mechanism according to the first embodiment.

The support member 16 is comprised of one or more rectangular-parallelepiped brackets, i.e. supports, 160 that are attached to the outer surface of the right sidewall 112 so as to project from the outer surface. In the first embodiment, two brackets 160 are attached to the outer surface of the right sidewall 112 so as to be aligned in the longitudinal direction of the base 114 with a space therebetween.

The brackets 160 have cylindrical through holes 161 coaxial to each other; each of the cylindrical through holes 161 has a center axis parallel to the longitudinal direction of the base 114.

The supporting member 16 also includes a push button 162, a button hole 163, an elastic member 164, and a lock member 165 provided in, for example, one of the brackets 160; one of the brackets 160 in which these members 162 to 165 are provided will be referred to as an adjusting bracket 160.

The elastic member 164 has a first end and a second end opposite to the first end. The first end of the elastic member 164 is fixed to a first portion FP of an inner periphery of the through hole 161 such that the second end of the elastic member 164 elastically pushes one side of the superelastic-alloy wire rod 141 fitted in the through hole 161 to a second portion SP of the inner periphery of the through hole 161; the one side faces the second end of the elastic member 164, and the first portion FP faces the second portion SP. This prevents movement of the predetermined length portion of the superelastic-alloy wire rod 141 in the longitudinal direction of the base 114.

The button hole 163 is formed from a top side of the adjusting bracket 160 so as to communicate with the through hole 161. The push button 162 is installed in the button hole 163 to face the elastic member 164 and to be movable in the length direction of the button hole 163. When a top end of the push button 162 is pushed down, the lock member 165 locks the pushed position of the push button 162. This causes a bottom end of the push button 162 to act on the elastic member 164 to reduce the pushing force of the elastic member 164 to the predetermined length portion of the superelastic-alloy wire rod 141. This permits the superelastic-alloy wire rod 141, i.e. the connecting wire 14, to be freely movable in the longitudinal direction of the base 114.

Referring to FIG. 1, the multijoint arm 12 is designed as a movement mechanism that movably supports the arm holder 11 according to external force applied to the arm holder 11. Specifically, the multijoint arm 12 has, for example, five rotational joints 31, 32, 33, 34, and 35 that provide five degrees of freedom.

Specifically, the multijoint arm 12 is comprised of a supporting base 41, a shoulder 42, a first arm member 43, and a second arm member 44.

The supporting base 41 is located on a floor F of an operating room to support the multijoint arm 12. For example, the supporting base 41 is equipped with casters (not shown) located at a bottom portion thereof, so that the supporting base 41 is easily movable on the floor F. The supporting base 41 also has a stopper (not shown) provided for each of the casters. A doctor or an assistant manipulates the stopper for each caster to stop the movement of the caster. This makes it possible to fixedly locate the supporting base 41 at a desired position of the floor F.

The joint 31 has a vertical axis orthogonal to, for example, the floor F, and is mounted on a first longitudinal end of a top of the supporting base 41 such that the vertical axis is orthogonal to the floor F.

On the joint 31, a first end of a link L is mounted. On a second end of the link L opposite to the first end, a shoulder portion 42 is mounted to extend upwardly from the second end of the link L such that the joint 32 is located at a top end of the shoulder portion 42. The shoulder portion 42 and the link L are rotatable about the vertical axis of the joint 31.

The first arm member 43 has a first end and a second end opposite to the first end. The joint 32 has a horizontal axis orthogonal to the vertical axis of the joint 31. The first end of the first arm member 43 is attached to the joint 32 and the shoulder portion 42 such that the first arm member 43 is swingable about the horizontal axis of the joint 32. The joint 33 having a horizontal axis parallel to the horizontal axis of the joint 32 is attached to the second end of the first arm member 43.

For example, the first arm member 43 is designed as a parallel link mechanism comprised of a set of first and second links 43a1 and 43a2. The first and second links 43a1 and 43a2 are capable of moving while keeping the first and second links 43a1 and 43a2 parallel to each other with a constant space therebetween.

A first end of the first link 43a1 is joined to the joint 32 to be swingable about the horizontal axis of the joint 32. A first end of the second link 43a2 is also joined to a pivot point P1 to be swingable about a horizontal axis of the pivot point P1 parallel to the horizontal axis of the joint 32; the pivot point P1 is attached to the second end of the link L. A second end of the first link 43a1, which is opposite to the first end, is joined to the joint 33 to be swingable about the horizontal axis of the joint 33. A second end of the second link 43a2, which is opposite to the first end, is also joined to a pivot point P2 to be swingable about a horizontal axis of the pivot point P2; the horizontal axis of the pivot point P2 is parallel to the horizontal axis of the joint 33.

The second arm member 44 has a first end and a second end opposite to the first end. The first end of the second arm member 44 is attached to the joint 33 such that the second arm member 44 is swingable about the horizontal axis of the joint 33. The joint 34 having a horizontal axis parallel to the horizontal axis of the joint 33 is attached to the second end of the second arm member 44.

For example, the second arm member 44 is designed as a parallel link mechanism comprised of a set of first and second links 44a1 and 44a2 configured to move while keeping the first and second links 44a1 and 44a2 in parallel to each other with a constant space therebetween. A first end of the first link 44a1 is joined to the joint 33 to be swingable about the horizontal axis of the joint 33. A first end of the second link 44a2 is also joined to the pivot point P2 to be swingable about the horizontal axis of the pivot point P2. A second end of the first link 44a1, which is opposite to the first end, is joined to the joint 34 to be swingable about the horizontal axis of the joint 34. A second end of the second link 44a2, which is opposite to the first end, is also joined to a pivot point P3 to be swingable about a horizontal axis of the pivot point P3; the horizontal axis of the pivot point P3 is parallel to the horizontal axis of the joint 34.

Note that the link L of the shoulder portion 42 connecting between the joint 32 and the pivot P1, and a link L11 connecting between the joint 33 and the pivot P2 are provided. The links L and L11 permit the parallel link mechanism of the first arm member 43 to move while maintaining a constant space therebetween. Similarly, there is a link L12 connecting between the joint 34 and the pivot P3. The links L11 and L12 permit the parallel link mechanism of the second arm member 44 to move while maintaining a constant space therebetween.

To the joint 34, the second end 114c of the base 114 of the arm holder 11 is attached via the joint 35 such that a vertical axis of the joint 35 is orthogonal to the horizontal axis of the joint 34. Specifically, the arm holder 11 is rotatable about the vertical axis of the joint 35. The first end 114b of the base 114 of the arm holder 11 is designed as a free end.

Between the second link 43a2 of the first arm member 43 and the joint 33, a spring 46 is provided, and, between the second link 44a2 of the second arm member 44 and the joint 33, a spring 47 is provided. The first end of the first link 43a1 of the first arm member 43 extends through the joint 32 to be far from the joint 32 by a preset length. To the extending end of the first link 43a1 of the first arm member 43, counterweights 48 are attached.

The springs 46 and 47 and the counterweights 48 serve as, for example, a biasing mechanism, and are operative to, when the forearm FA of a doctor is mounted on the mount surface 114a of the arm holder 11, apply counterbalance force to the arm holder 11 and the multijoint arm 12.

Specifically, biasing force from the springs 46 and 47 and the counterweights 48 biases the arm holder 11 upwardly. The biasing force applied to the arm holder 11 counterbalances the sum of the weight of the arm holder 11, the weight of, for example, the forearm FA held by the arm holder 11, and the weight of the multijoint arm 12. The sum of these weights will be referred to as an arm total weight hereinafter.

This balance supports the forearm FA mounted on the arm holder 11.

Note that the biasing force should be ideally counterbalanced to the arm total weight.

However, the hand HA of the forearm FA of a doctor normally performs surgical operations to an affected site of a living body, such as a patient, from above. Thus, in consideration of this matter, the biasing force is determined to bias, with very weak force, the arm holder 11 in the upward direction. Note that the upward direction means a direction relative to the affected site which is receiving surgical treatment from the hand HA of a doctor, and thereby treatment of the affected site can be safely performed while the arm holder 11 is prevented from being unintentionally lowered. At least one of the springs 46 and 47 can be eliminated if the balance of force is established with the use of only the counterweights 48. One of various types of measures for biasing the arm holder 11 can be used.

To the joint 31, a brake, such as an electromagnetic brake, 31A is attached for reducing rotation of the shoulder portion 42 relative to the supporting base 41 around the vertical axis of the joint 31.

Like the joint 31, to the joint 32, a brake, such as an electromagnetic brake, 32A is attached for reducing rotation of the first arm member 43 relative to the shoulder portion 42 around the horizontal axis of the joint 32.

Additionally, to the joint 33, a brake, such as an electromagnetic brake, 33A is attached for reducing rotation of the second arm member 44 relative to the joint 33 around the horizontal axis of the joint 33.

Each of the brakes 31A to 33A is communicably connected to the controller 13, and can be controlled by the controller 13. The brakes 31A to 33A serve as, for example, a first limiter for limiting movement of the arm holder 11 and for releasing limit of movement of the arm holder 11.

The controller 13 is, for example, designed as an electronic control circuit equipped with, for example, a CPU 131, a ROM 132, and a RAM 133.

The CPU 131 is programmed to set an operation mode of the arm support apparatus 1 to, for example, one of a lock mode and a free mode.

The lock mode is designed assuming that a doctor tries to perform surgical operations using the hand HA of the dominant arm of a doctor while the forearm FA is free from the arm holder 11.

Specifically, in the lock mode, the CPU 131 activates the brakes 31A, 32A, and 33A to stop movement of the corresponding joints 31, 32, and 33, respectively. This results in prevention of movement of the arm holder 11. Thus, in the lock mode, the doctor can freely move the dominant arm to perform fine surgical operations to the affected site using the hand HA of the dominant arm while movement of the arm holder 11 is locked. In the lock mode, because no brakes are provided for the respective joints 34 and 35, the doctor can easily turn the arm holder 11 around the vertical axis of the joint 35, and turn the arm holder 11 around the horizontal axis around the joint 34.

The free mode is designed assuming that a doctor tries to make the arm holder 11 follow movement of the dominant arm. That is, in the free mode, the CPU 131 deactivates the brakes 31A, 32A, and 33A to allow movement of the corresponding joints 31, 32, and 33, respectively. This results in free movement of the arm holder 11. Because the force applied from the arm holder 11 to the forearm FA is very weak, and slide resistance of each of the brakes 31A, 32A, and 33A is small, the doctor can easily move the arm holder 11 to follow movement of the dominant arm using weak force applied to the arm holder 11.

As described above, the CPU 131 is capable of selecting one of the lock mode and the free mode. In the first embodiment, there can be various measures to instruct the CPU 131 to select one of the lock mode and the free mode. For example, as an example of the measures according to the first embodiment, the arm support apparatus 1 includes a mode selection switch 134 designed as a foot switch and communicably connected to the CPU 131. Specifically, the CPU 131 can select one of the lock mode and the free mode according to how a doctor or an assistant depresses the mode selection switch 134.

Next, operations of the arm support apparatus 1 according to the first embodiment will be described when the free mode is selected as the operation mode of the arm support apparatus 1.

In the free mode, as described above, biasing force, which is applied to the arm holder 11 in the upward direction, is enough to balance the arm total weight. This feature makes the arm holder 11 easily follow movement of the doctor's dominant arm whose forearm FA is mounted on the arm holder 11 in the upward or downward direction. This results in maintenance of the mounted state of the forearm FA on the arm holder 11.

As described above, the arm support apparatus 1 includes a second limiter that is comprised of the following configurations:

(1) The left and right sidewalls 111 and 112 limit the forearm FA mounted on the base 114 of the arm holder 11 from moving outwardly away from the base 114 in the lateral direction of the base 114.

(2) The elbow-position limiter 114 limits the forearm FA mounted on the base 114 from moving outwardly away from the second end 114c in the backward direction relative to the arm holder 11.

These configurations of the second limiter make the arm holder 11 follow movement of the doctor's dominant arm whose forearm FA is mounted on the arm holder 11 in the horizontal direction, i.e. the leftward or rightward direction, or the backward direction.

In addition, the grip 15 of the grippable member GM is located to be grippable by a part, i.e. the little finger, of the hand HA of the forearm FA mounted on the base 114 of the arm holder 11, and the grip 15 is coupled to the arm holder 11 via the connecting wire 14.

With this configuration, while the grip 15 is gripped by the little finger of the doctor's hand HA, the doctor moves the dominant arm, i.e. the forearm FA, in the frontward direction. This causes the grip 15 and the connecting wire 14 to pull the arm holder 11 in the frontward direction, resulting in the arm holder 11 following movement of the doctor's dominant arm in the front direction.

That is, in the free mode, the arm support apparatus 1 makes it possible for the doctor to pull the grip 15 by the little finger of the hand HA in the frontward direction to cause the arm holder 11 to follow movement of the hand HA in the frontward direction. In the free mode, the arm support apparatus 1 makes it possible for the doctor to merely move the dominant arm, i.e. the forearm FA, in the upward direction, the downward direction, the rightward direction, the leftward direction, or the backward direction to cause the arm holder 11 to follow movement of the dominant arm in the same direction.

Thus, the arm support apparatus 1 achieves an advantage of making the arm holder 11 follow lightly and stably movement of the doctor's dominant arm, i.e. the forearm FA, without the forearm FA being fixed to the arm holder 11 even if the friction coefficient between the forearm FA and the arm holder 11 is set to a lower value.

In addition, the connecting wire 14 is configured to be deformable according to external force applied thereto or to the grip 15, thus supporting the grip 15 such that the position of the grip 15 is changeable. The connecting wire 14 is also configured to be non-extendable in the front direction. This increases the freedom of motion of the writs of the doctor's dominant arm with a higher following capability of the arm holder 11 with respect to movement of the doctor's dominant arm as compared with the structure that the position of the grip 15 is fixed with respect to the arm holder 11. Additionally, because the grip 15 is configured to be non-extendable in the front direction, it is possible to increase the following ability of the arm holder 11 with respect to the pulling operation of the grip 15.

The connecting wire 14 is comprised of an elastic deformation portion deformed when an external force is applied thereto or to the grip 15. Thus, the elastically deformed elastic deformation portion of the connecting wire 14 returns to an original shape, i.e. an initial shape, when external force is removed therefrom. This makes it possible for the doctor to directly understand the position of the grip 15.

The elastic deformation portion is made from a super-elastic alloy having little aging deterioration and little plastic deformation, making it possible to improve the durability and reliability of the connecting wire 14.

The connecting wire 14 is also comprised of a deformation holding portion that is elastically deformed when an external force is applied thereto or to the grip 15, and that holds the deformed shape. This makes it possible for the doctor to easily change the original position, i.e. initial position, of the grip 15 while no external force is applied to the grip 15 to a desired position that is close to the hand HA, i.e. the little finger.

The arm support apparatus 1 includes the support member 16 for supporting the grip 15 while having a capability to adjust the position of the grip 15 relative to the elbow-portion limiter 113, i.e. the elbow E of the forearm FA whose location is limited by the elbow-position limiter 113 in the longitudinal direction of the base 114. This allows a doctor to easily adjust the position of the grip 15 relative to the elbow-portion limiter 113 in the longitudinal direction of the base 114 according to the length of the doctor's forearm FA in the longitudinal direction of the base 114. This reduces redundant play in the relative distance between the grip 15 and the arm holder 11 in order to allow variations in length of the different doctor's forearms FA, making it possible to increase the following ability of the arm holder 11 with respect to the pulling operation of the grip 15.

### Second Embodiment

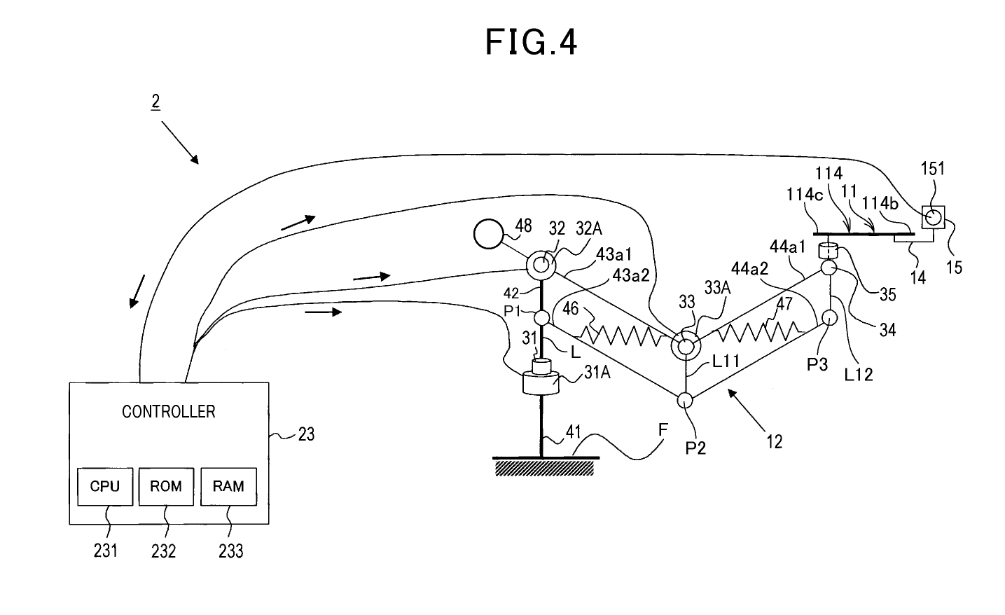

An arm support apparatus 2 according to a second embodiment of the present disclosure will be described hereinafter with reference to FIGS. 4 to 6.

The structure and functions of the arm support apparatus 2 are slightly different from those of the arm support apparatus 1 by the following points. So, the different points will be mainly described hereinafter.

The arm support apparatus 2 includes a grip sensor 151 in place of the mode selection switch 134. The grip sensor 151 is communicably connected to a controller 23. The grip sensor 151 is operative to measure external force applied to the grip 15, such as force gripping the grip 15, and output a force signal indicative of the measured value of the external force applied to the grip 15 to the controller 23.

For example, in the second embodiment, the grip sensor 151 is installed in the grip 15. A pressure sensor for measuring pressure applied thereto is used as the grip sensor 151. One of various types of sensors and switches are used in place of the pressure sensor. The various types of sensors and switches include: a sensor for detecting that something contacts to the grip 15, a sensor for detecting its strain caused when the grip 15 is gripped by something, and a button switch for outputting a signal when the grip 15 is gripped so that the button switch is turned on.

As described above, the arm support apparatus 2 includes the controller 23 in place of the controller 13.

Like the first embodiment, the controller 23 is, for example, designed as an electronic control circuit equipped with, for example, a CPU 231, a ROM 232, and a RAM 233.

The CPU 231 is programmed to control the brakes 31A, 32A, and 33A according to the force signal indicative of the external force applied to the grip 15, thus setting the operation mode of the arm support apparatus 2 to, for example, one of the lock mode and the free mode.

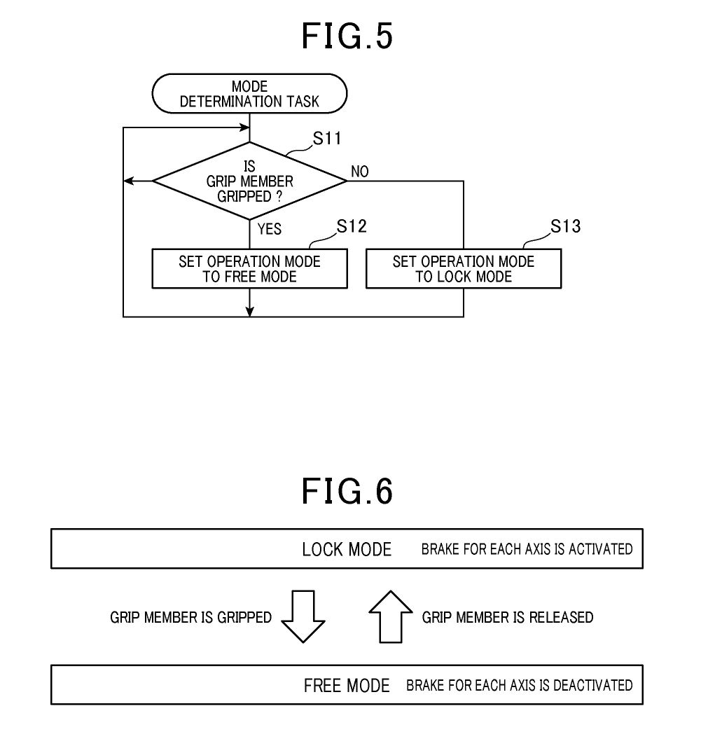

Next, operations of a mode determination task executed by the controller 23 will be described hereinafter with reference to FIG. 5. The mode determination task is cyclically performed by the CPU 231 while the arm support apparatus 1 is powered on.

In step S11 of FIG. 5, the controller 23, i.e. the CPU 231, determines whether the grip 15 is gripped based on the force signal sent from the grip sensor 151. Specifically, in step S11, the CPU 231 determines whether the external force applied to the grip 15 included in the force signal is equal to or greater than a threshold value.

Upon determination that the external force applied to the grip 15 is equal to or greater than the threshold value (YES in step S11), the CPU 231 determines that the grip 15 is gripped, so that the procedure of the mode determination task proceeds to step S12.

In step S12, the CPU 231 determines the operation mode of the arm support apparatus 2 to the free mode in step S12, returning to step S11. Specifically, in step S12, the CPU 231 deactivates all the brakes 31A, 32A, and 33A to allow free movement of the corresponding joints 31, 32, and 33, respectively, thus shifting the operation mode of the arm support apparatus 2 to the free mode.

Otherwise, upon determination that the external force applied to the grip 15 is smaller than the threshold value (NO in step S11), the CPU 231 determines that the grip 15 is not gripped by anyone. Then, the procedure of the mode determination task proceeds to step S13.

In step S13, the CPU 231 activates all the brakes 31A, 32A, and 33A to lock movement of the corresponding joints 31, 32, and 33, respectively, thus shifting the operation mode of the arm support apparatus 2 to the lock mode.

Specifically, as illustrated in FIG. 6, doctor's grip of the grip 15 shifts the operation mode of the arm support apparatus 2 to the free mode, so that the free mode is maintained while the grip 15 is gripped. Thereafter, doctor's release of the grip 15 shifts the operation mode of the arm support apparatus 2 to the lock mode.

Other functions of the arm support apparatus 2 are substantially identical to those of the arm support apparatus 1.

As described above, the arm support apparatus 2 is configured such that the grip sensor 151 determines whether the grip 15 is gripped, and the controller 23 is configured to switch the operation mode of the arm support apparatus 2 to one of the free mode and the lock mode according to whether the grip 15 is gripped. This configuration allows the doctor to easily apply force to the grip 15 using a part of the hand HA, i.e. the little finger of the hand HA, thus easily switching the operation mode of the arm support apparatus 2 to one of the free mode and the lock mode.

The configuration of the arm support apparatus 2, which switches the operation mode of the arm support apparatus 2 to the free mode while the grip 15 is gripped, makes it possible to more safely move the arm holder 11.

In the second embodiment, the controller 23 switches the operation mode of the arm support apparatus 2 to the lock mode based on whether the external force applied to the grip 15 is equal to or greater the threshold value, but the present disclosure is not limited thereto. Specifically, the controller 23 can switch the operation mode of the arm support apparatus 2 to the lock mode each time the grip 15 is gripped.

### Third Embodiment

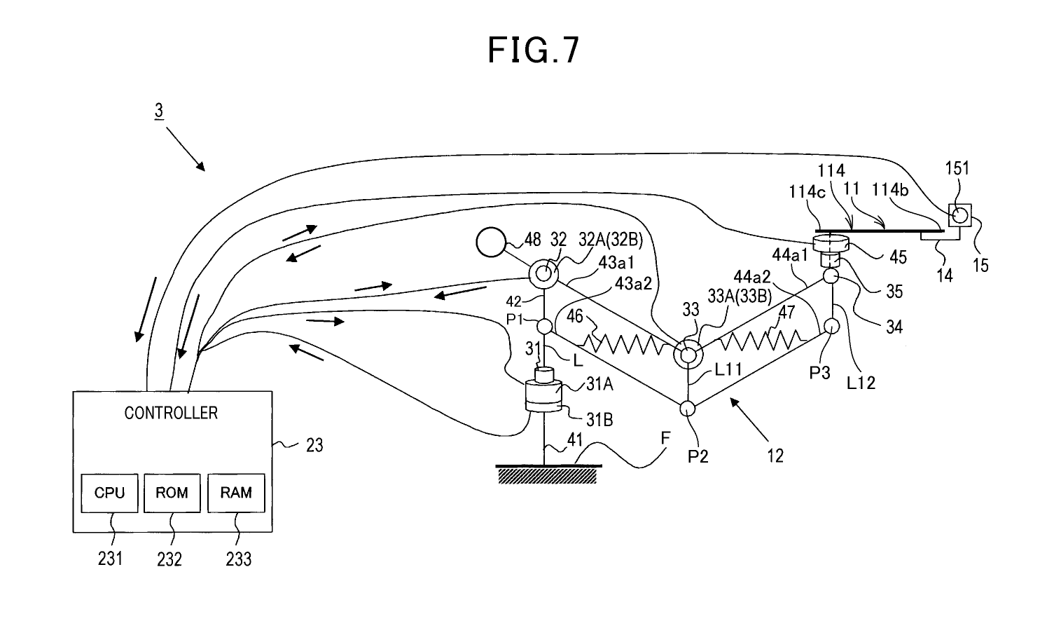

An arm support apparatus 3 according to a third embodiment of the present disclosure will be described hereinafter with reference to FIGS. 7 to 9.

The structure and functions of the arm support apparatus 3 are slightly different from those of the arm support apparatus 2 by the following points. So, the different points will be mainly described hereinafter.

The arm support apparatus 3 further includes encoders 31B, 32B, and 33B attached to the respective joints 31, 32, and 33. The encoder 31B is operative to measure an amount of rotation of the shoulder portion 42 relative to the supporting base 41. The encoder 32B is operative to measure an amount of rotation of the first arm member 43 relative to the shoulder portion 42. The encoder 33B is operative to measure an amount of rotation of the second arm member 44 relative to the joint 33.

Each of the encoders 31 B to 33B is communicably connected to the controller 23, and operative to output a measurement signal indicative of the corresponding measured amount of rotation to the controller 23.

The arm support apparatus 3 further includes a force sensor 45 so that the arm holder 11 is joined to the joint 35 via the force sensor 45 to be rotatable about the vertical axis of the joint 35.

The force sensor 45 is communicably connected to the controller 23. The force sensor 45 is operative to measure, as force data applied to the arm holder 11, at least one of first force, second force, and third force respectively applied to the arm holder 11 in a first axis, a second axis, and a third axis. The first, second, and third axes are defined at, for example, a predetermined point of the arm holder 11 through which an extending line of the vertical axis of the joint 35 passes. The first axis corresponds to, for example, the vertical axis, i.e. the upward and downward axis. The external force applied to the arm holder 11 represents whether the doctor's forearm FA is mounted on the arm holder 11.

The force sensor 45 is also operative to measure, as torque data applied to the arm holder 11, at least one of first torque about the first axis, second torque about the second axis, and third torque about the third axis.

The force sensor 45 is operative to output the measured force data and torque data to the controller 23. One of various types of sensors are used in place of the force sensor 45. The various types of sensors include: a sensor for detecting that something contacts to the arm holder 11, a sensor for detecting its train caused when something is mounted on the arm holder 11, and a pressure sensor for detecting pressure on the arm holder 11 due to something mounted on the arm holder 11.

The CPU 231 is programmed to control the brakes 31A, 32A, and 33A according to the force signal sent from the grip sensor 151, the measured force data and torque data sent from the force sensor 45, and the measurement signal sent from each of the encoders 31B, 32B, and 33B.

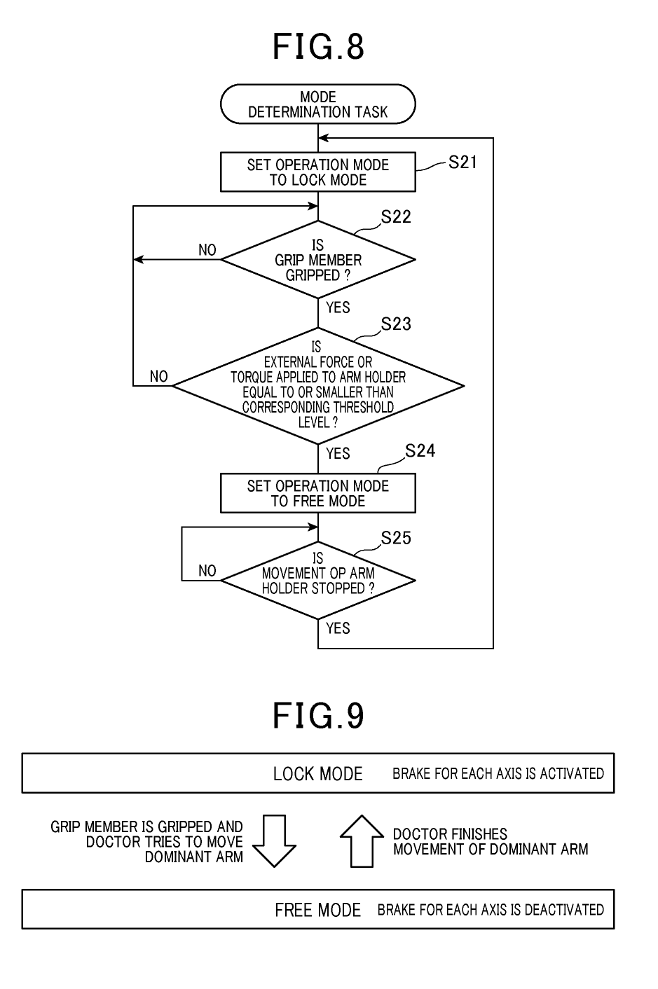

Next, operations of a mode determination task executed by the controller 23 will be described hereinafter with reference to FIG. 8. The mode determination task is cyclically performed by the CPU 231 while the arm support apparatus 1 is powered on.

In step S21 of FIG. 8, the controller 23, i.e. the CPU 231, sets the operation mode of the arm support apparatus 3 to the lock mode. The operation in step S21 is identical to that in step S13 in FIG. 5.

Next, in step S22 of FIG. 5, the CPU 231 determines whether the grip 15 is gripped based on the force signal sent from the grip sensor 151. The operation in step S22 is identical to that in step S11 in FIG. 5.

Until it is determined that the grip 15 is not gripped by anyone (NO in step S22), the CPU 231 repeats the determination in step S22.

Otherwise, it is determined that the grip 15 is gripped (YES in step S22), the procedure proceeds to step S23.

In step S23, the CPU 231 determines, based on the measured force data and torque data sent from the force sensor 45, whether the following first and second conditions are satisfied:

(1) Force applied to the arm holder 11 is equal to or smaller than a threshold level F for 200 ms.

(2) Torque applied to the arm holder 11 has been equal to or smaller than a threshold level Tr for 200 ms; the threshold level Tr corresponds to the first threshold level F.

The threshold level F is set to be equal to 1.0 kgf (9.8 N), and the threshold level Tr is set to be equal to 5.0 kg·cm (49 N·cm).

The state in which at least one of the first and second conditions is satisfied represents that the doctor is likely to put muscle to the dominant arm to move the dominant arm. Specifically, when the doctor tries to positively make the arm holder 11 follow movement of the dominant arm, the doctor supports the dominant arm by its muscle first, and applies force or torque to the arm holder 11 via the dominant arm next. When the doctor supports the dominant arm by its muscle, force applied to the arm holder 11 from above becomes temporarily be less than the threshold level F, such as 1.0 kgf, or torque applied to the arm holder 11 becomes temporarily be less than the threshold level T, such as 5.0 kg·cm. Thus, when at least one of force and torque applied to the arm holder 11 is equal to or smaller than a corresponding one of the threshold level F and the threshold level T for 200 ms, it is determined that the doctor is trying to move the dominant arm.

Upon determination that neither the first condition nor the second condition is satisfied (NO in step S23), the CPU 231 returns to step S22, and repeats the operations from step S22. Otherwise, upon determination that at least one of the first condition nor the second condition is satisfied (YES in step S23), the CPU 231 sets the operation mode of the arm support apparatus 3 to the free mode in step S24. The operation in step S24 is identical to that in step S12 in FIG. 5.

Next, the CPU 231 determines whether movement of the arm holder 11 is stopped based on the measurement signal indicative of the corresponding measured amount of rotation sent from each of the encoders 31B, 32B, and 33B in step S25. In other words, the CPU 231 determines whether the doctor tries to finish movement of the arm holder 11 at a desired position to thereby fix the arm holder 11 to the corresponding position, based on the measurement signal indicative of the corresponding measured amount of rotation sent from each of the encoders 31B, 32B, and 33B in step S25.

Specifically, in step S25, the CPU 231 calculates the movement speed of the arm holder 11 based on the measurement signal indicative of the corresponding measured amount of rotation sent from each of the encoders 31B, 32B, and 33B. Then, in step S25, the CPU 231 determines whether the calculated movement speed of the arm holder 11 has been equal to or lower than a preset threshold level V of, for example, 1 mm/s for 100 ms.

In other words, the CPU 231 determines whether rotation of each of the shoulder portion 42, the first arm member 43, and the second arm member 44 around a corresponding one of the joints 31, 32, and 33 is stopped in step S25.

Until it is determined that the calculated movement speed of the arm holder 11 has not been equal to or lower than the preset threshold level V for 100 ms (NO in step S25), the CPU 231 repeats the determination in step S25.

Otherwise, it is determined that the calculated movement speed of the arm holder 11 has been equal to or lower than the preset threshold level V for 100 ms (YES in step S25), the CPU 231 determines that the doctor tries to finish movement of the arm holder 11 at a desired position to thereby fix the arm holder 11 to the corresponding position.

Then, the CPU 231 returns to step S21, and sets the operation mode of the arm support apparatus 3 to the lock mode in step S21.

Specifically, as illustrated in FIG. 9, doctor's grip of the grip 15 and doctor's attempt to move the dominant arm shift the operation mode of the arm support apparatus 3 to the free mode. Thereafter, doctor's finish of movement of the dominant arm shifts the operation mode of the arm support apparatus 3 to the lock mode.

Other functions of the arm support apparatus 3 are substantially identical to those of the arm support apparatus 1.

As described above, the arm support apparatus 3 is configured to shift the operation mode to the lock mode when determining, based on the measured amount of rotation sent from each of the encoders 31B, 32B, and 33B, that the arm holder 11 is stopped.

The arm support apparatus 3 is also configured to shift the operation mode to the free mode when determining, based on the force signal sent from the grip sensor 151 and the measured force data and torque data sent from the force sensor 45, that the following conditions are satisfied:

(i) The grip 15 is gripped

(ii) The doctor tries to finish movement of the dominant arm.

This configuration permits the doctor to switch the operation mode of the arm support apparatus 3 between the lock mode and the free mode using intuitive operations.

For example, while gripping the grip 15, the doctor merely has one action to float the dominant arm, making it possible to shift the operation mode of the arm support apparatus 3 to the free mode. Thereafter, the doctor merely holds the dominant arm at standstill, making it possible to shift the operation mode of the arm support apparatus 3 to the lock mode.

In addition, for replacement of a surgical tool gripped by the hand HA, the doctor naturally releases the grip 15. For this reason, the doctor merely floats the dominant arm while the grip 15 is not gripped thereby, resulting in shift of the operation mode to the lock mode, thus fixing the position of the arm holder 11.

The present disclosure is not limited to the aforementioned embodiments, and various modifications of each embodiment can be performed within the scope of the present disclosure.

For example, the arm support apparatus according to each of the first to third embodiments is equipped with the support member 16 for supporting the grip 15 while having a capability to adjust the position of the grip 15 relative to the elbow-portion limiter 113 in the longitudinal direction of the base 114, but the present disclosure is not limited thereto. Specifically, the arm support apparatus can be equipped with an adjusting mechanism. The adjusting mechanism is configured to adjust the position of the elbow-portion limiter 113 relative to the position of the grip 15 in the longitudinal direction of the base 114 in place of or in addition to adjustment of the position of the grip 15 relative to the elbow-portion limiter 113 in the longitudinal direction of the base 114.

In the arm support apparatus according to each of the first to third embodiments, the left and right sidewalls 111 and 112, and the elbow-portion limiter 113 serve as a second limiter that limits movement of the forearm FA of the dominant arm mounted on the arm holder 11 in both the backward direction and the leftward or rightward direction.

However, the present disclosure is not limited to the configurations.

Specifically, any structures that limit the forearm FA mounted on the base 114 of the arm holder 11 from outwardly moving from the base 114 in each of the backward direction, the leftward direction, and the rightward direction can be included in the concept of the second limiter according to the present disclosure.

The arm support apparatus according to each of the first to third embodiments is equipped with the elbow-portion limiter 113. The elbow-portion limiter 113 is made from a high-rigidity material and adapted to limit the forearm FA mounted on the base 114 from moving outwardly away from the second end 114c in the longitudinal direction of the base 114 relative to the arm holder 11. The present disclosure is however not limited to the structure.

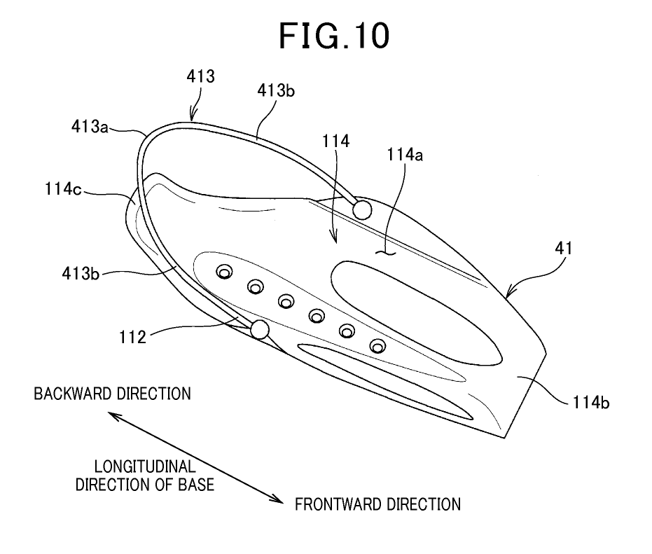

FIG. 10 schematically illustrates an arm holder 41 according to a modification of each of the first to third embodiments. Note that, in FIG. 10. the connecting wire 14, the grip 15, and the support member 16 are omitted in illustration for simply illustration of the arm holder 41.

Referring to FIG. 10, the arm holder 41 includes an elbow-position limiter 413 made from, for example, a string member. The elbow-position limiter 413 has an arched shape. Specifically, the elbow-position limiter 413 includes a substantially U-shaped curved portion 413a, a first leg portion 413b, and a second leg portion 413c. The first leg portion 413b has a first end attached to the left sidewall 111, and the second leg portion 413c has a first end attached to the right sidewall 112.

The first leg portion 413b has a second end opposite to the first end thereof, and the second end is continuously joined to one end of the curved portion 413a. The second leg portion 413c has a second end opposite to the first end thereof, and the second end is continuously joined to the other end of the curved portion 413a. The curved portion 413a is located above the second end 114c to which a part of the elbow E is fittable.

Specifically, when the forearm FA is mounted on the mount surface 114a while the hand HA of the dominant arm projects from the first end 114b of the base 114, a lower portion of the elbow E is mounted on the second end 114c of the mount surface 114a with an upper portion of the elbow E is fitted in the elbow-position limiter 413. This limits the forearm FA mounted on the mount surface 114a of the base 114 from moving outwardly away from the second end 114c in the longitudinal direction of the base 114 relative to the arm holder 11.

The elbow-position limiter 413 is configured to be deformable when external force is applied thereto. The elbow-position limiter 413 is also configured to be non-extendable in the longitudinal direction of the base 114. In other words, the length of the elbow-position limiter 413 does not extend, which is different from the length of a material with a low level of hardness, such as rubber.

For example, the respective ends of the elbow-position limiter 413 are fixed to the left and right sidewalls 111 and 112 so as not to be pivotable about the fixed portions, and the elbow-position limiter 413 is deformed to be bent in a given direction in which external force is applied. For example, while the elbow-position limiter 413 is deformed by external force, when the external force is removed, the deformed elbow-position limiter 413 is configured to return to its original position. The elbow-position limiter 413 can be made from a super-elastic alloy.

As described above, the elbow-position limiter 413 is configured to be deformable depending on how the dominant arm is bent. This configuration makes it possible for the elbow E to be easily fittable to the elbow-position limiter 413 independently of whether the dominant arm is bent or stretched. Thus, the elbow-position limiter 413 permits various postures of the dominant arm while the forearm FA is fitted thereto.

Note that, as described above, the arm holders 11 according to the first to third embodiments are designed for doctors whose dominant arms are right arms, but naturally the arm holders 11 according to the first to third embodiments can be designed for doctors whose dominant arms are left arms. Because the elbow-position limiter 413 is designed to be deformable when external force is applied thereto, products of the arm holders 11 according to the first to third embodiments can be commonly used both for the arm holders 11 for doctors whose dominant arms are right arms and for the arm holders 11 for doctors whose dominant arms are left arms.

The connecting wire 14 according to each of the first to third embodiments is comprised of the superelastic-alloy wire rod 141 serving as an elastic deformation portion, and the mild-steel wire rod 142 serving as a deformation holding portion, but the present disclosure is not limited thereto. For example, the elastic deformation portion of the connecting wire 14 can be made from an elastic member, which has little plastic deformation and is easily elastically deformable. The deformation holding portion of the connecting wire 14 can be made from a material, which has easy plastic deformation and is difficult to be damaged. The connecting wire 14 can be comprised of the elastic deformation portion without the deformation holding portion, or can be comprised of the deformation holding portion without the elastic deformation portion.

The arm support apparatuses according to the first to third embodiments are applied for supporting one arm of a doctor who is performing surgical operations, but the present disclosure is not limited to this application. Specifically, the arm support apparatuses according to the first to third embodiments can be applied for supporting one arm of an operator who performs operations to, for example, manufacture various machines or devices, such as precise machines of devices.

One or more functions performed by one element of each of the arm support apparatuses 1 to 3 can be shared to plural elements of a corresponding one of the arm support apparatuses 1 to 3. Plural functions included in plural elements of each of the arm support apparatuses 1 to 3 perform can be integrally installed in one element of a corresponding one of the arm support apparatuses 1 to 3.

A part of the structure of each of the arm support apparatuses 1 to 3 can be replaced with a known structure as long as the remaining structure of each of the arm support apparatuses 1 to 3 is capable of addressing the circumstances described in SUMMARY. A part of the structure of each of the arm support apparatuses 1 to 3 can be eliminated as long as the remaining structure of each of the arm support apparatuses 1 to 3 is capable of addressing the circumstances described in SUMMARY.

At least part of the structure of each of the arm support apparatuses 1 to 3 can be added to the structures of the other arm support apparatuses, or can be replaced with at least part of the structure of each of the other arm support apparatuses.

The present disclosure includes an improved controller constituting at least one of the arm support apparatuses 1 to 3, and an improved program including a set of computer program instructions that causes a computer to perform one or more tasks. The present disclosure includes an improved storage medium storing therein the program, and an improved method of how to shift an operation mode of at least one of the arm support apparatuses 1 to 3 to another operation mode.

While illustrative embodiments of the present disclosure have been described herein, the present disclosure is not limited to the embodiments described herein, but includes any and all embodiments having modifications, omissions, combinations (e.g., of aspects across various embodiments), adaptations and/or alternations as would be appreciated by those in the art based on the present disclosure. The limitations in the claims are to be interpreted broadly based on the language employed in the claims and not limited to examples described in the present specification or during the prosecution of the application, which examples are to be construed as non-exclusive.

What is claimed is:

1. An arm support apparatus for supporting an arm of an operator, the arm support apparatus comprising:
a longitudinal mount on which a part of the arm of the operator is mountable, the longitudinal mount having a first end, a second end opposite to the first end in a longitudinal direction thereof, and a predetermined lateral width;
a support member that has at least one joint and supports the longitudinal mount to be movable based on bending of the at least one joint;
a first limiter that limits motion of the at least one joint to limit movement of the longitudinal mount when an operation mode of the arm support apparatus is set to a first mode,
the first limiter releasing limit of motion of the at least one joint to release limit of movement of the longitudinal mount when the operation mode of the arm support apparatus is set to a second mode;
a biasing mechanism that applies biasing force to the longitudinal mount to bias the longitudinal mount upwardly when the operation mode of the arm support apparatus is set to the second mode;
a grippable member connected to the longitudinal mount and located such that a part of the grippable member is grippable by a hand of the arm when the part of the arm is mounted on the longitudinal mount, the hand being located to be closer to the first end of the longitudinal mount than to the second end of the longitudinal mount; and
a second limiter that limits movement of the part of the arm mounted on the longitudinal mount in both a first direction and a second direction,
the first direction being away from the second end of the longitudinal mount in the longitudinal direction, the second direction being parallel to the lateral width of the longitudinal mount and perpendicular to the longitudinal direction.

2. The arm support apparatus according to claim 1, wherein the grippable member comprises:
a grip serving as the part of the grippable member and located to be grippable by the hand of the arm when the part of the arm is mounted on the longitudinal mount; and
a connecting member connecting the longitudinal mount and the grip,
the connecting wire being configured to be deformable according to external force applied thereto or the grip, thus supporting the grip such that a position of the grip is changeable relative to the longitudinal mount.

3. The arm support apparatus according to claim 1, wherein the grippable member comprises:
a grip serving as the part of the grippable member and located to be grippable by the hand of the arm when the part of the arm is mounted on the longitudinal mount; and
a connecting member connecting the longitudinal mount and the grip,
the connecting member comprising an elastic deformation portion configured to be deformable when external force is applied to the elastic deformation portion or the grip.

4. The arm support apparatus according to claim 3, wherein the elastic deformation portion is made from a super-elastic alloy.

5. The arm support apparatus according to claim 1, wherein the grippable member comprises:
a grip serving as the part of the grippable member and located to be grippable by the hand of the arm when the part of the arm is mounted on the longitudinal mount; and
a connecting member connecting the longitudinal mount and the grip,
the connecting member comprising a deformation holding portion configured to:
be deformable when external force is applied to the elastic deformation portion or the grip, and
hold a deformed shape thereof.

6. The arm support apparatus according to claim 1, further comprising:
an adjusting member that adjusts a position of one of the part of the arm limited in movement by the first limiter and the grip relative to a portion of the other of the arm and the grip in the longitudinal direction.

7. The arm support apparatus according to claim 1, further comprising:
a grip determiner that determines whether the grip is gripped; and
an operation mode setting unit that sets the operation mode of the arm support apparatus to one of the first mode and the second mode according to a determination result by the grip determiner.

8. The arm support apparatus according to claim 7, further comprising:
a movement determiner that determines whether movement of the longitudinal mount is stopped; and
a mount determiner that determines whether the part of the arm is mounted on the longitudinal mount,
wherein the operation mode setting unit is adapted to:
set the operation mode of the arm support apparatus to the first mode when it is determined that movement of the longitudinal mount is stopped; and
set, when it is determined that grip is gripped, the operation mode of the arm support apparatus to the second mode according to a determination result by the mount determiner.

9. The arm support apparatus according to claim 1, wherein the second limiter comprises an arched member attached to the longitudinal mount such that the arched member is located above the second end of the longitudinal mount, an elbow of the arm mounted on the longitudinal mount being fittable to the arched member.

10. The arm support apparatus according to claim 1, wherein:
the arched member comprises a curved strip member.

11. The arm support apparatus according to claim 10, wherein the strip member is configured to be deformable when external force is applied thereto, and to be non-extendable in a longitudinal direction thereof.

12. The arm support apparatus according to claim 10, wherein the strip member is configured to be elastically deformable when external force is applied thereto.

13. The arm support apparatus according to claim 9, wherein the strip member is made from a super-elastic alloy.
